(19) United States
(12) Patent Application Publication
(10) Pub. No.: US 2010/0087331 A1
(43) Pub. Date: Apr. 8, 2010

(54) METHOD OF NUCLEIC ACID ANALYSIS

(75) Inventors: Tamara Maes, Barcelona (ES); Olga Durany Turk, Barcelona (ES); Elena Aibar Duran, Barcelona (ES)

Correspondence Address: Workman Nydegger;1000 Eagle Gate Tower, 60 East South Temple, Salt Lake City, UT, 84111, US

(73) Assignee: ORYZON GENOMICS, S.A., Barcelona (ES)

(21) Appl. No.: 12/571,099

(22) Filed: Sep. 30, 2009

Related U.S. Application Data
(63) Continuation of application No. PCT/EP2008/053752, filed on Mar. 28, 2008.

(30) Foreign Application Priority Data
Mar. 30, 2007 (ES) ES200700966

(51) Int. Cl. C40B 30/04; C12Q 1/68

(52) U.S. Cl. 506/9; 435/6

(57) ABSTRACT

A method of nucleic acid analysis includes the stages of synthesizing a first complementary DNA strand from a messenger RNA using compound primers, synthesizing a second DNA strand, labeling by in vitro transcription of an RNA polymerase, and determining the presence of splicing events in the sample. The present invention has application, for example, in analyzing differential splicing events and in diagnosing diseases.

### CROSS-REFERENCE TO RELATED APPLICATIONS

This application is a continuation in part of PCT Application serial number PCT/EP2008/053752, titled “Method of Nucleic Acid Analysis,” filed Mar. 28, 2008, which claims the benefit of Spain Application No. 200700966, filed on Mar. 30, 2007, both of which are hereby incorporated herein by reference in their entirety.

### BACKGROUND OF THE INVENTION

1. The Field of the Invention

The present invention relates to the field of molecular biology. In particular, the present invention relates to a method of nucleic acid analysis that can be used to analyze the presence of alternative splicing events in a sample.

2. Related Technology

For decades RNA molecules have been regarded as little more than DNA messengers; regarded as simple intermediaries between the genetic code and the manufacture of proteins in the cell. However, research carried out in recent years has established that certain RNA molecules perform a much more important role in the cell.

An interesting phenomenon related to RNA is the splicing it undergoes before it becomes the final mRNA molecule that will be translated into a peptide. The process of splicing generally includes obtaining different mRNAs from the same primary transcript by alternating the intron splicing options. As a result of this process, each of the mRNAs obtained contains different exons of the gene from which it has been transcribed.

Though it was first thought that splicing was intended simply for the removal of non-coding introns from the primary transcript and subsequent joining together of exons, later it was observed that it was a gene regulation mechanism by means of which the cell could synthesize different proteins from a single RNA depending on a series of factors that dictate how splicing should be performed.

It is now known that splicing processes are of importance in regulating cellular processes as well as in the development of some diseases. It can be the case that a mutation in the gene results in a change in one of the splicing locations, which will give rise to reading frame shift mutations or the introduction of premature stop codons. Thus, for example, it is possible to speak of differential splicing, in which RNA molecules are observed that have been subjected to a different processing between the healthy state and the diseased state.

There is a growing interest in techniques that allow the study of RNA. However, one of the main problems that researchers are facing is that on many occasions the amount of RNA sample available to them is limited. For this reason a series of technologies have been developed to enable the amount of RNA obtainable from a sample to be increased.

One of the protocols used is linear amplification using oligo-dT primers, or Eberwine method (Van Gelder R N, von Zastrow M E, Yool A, Dement W C, Barchas J D, Eberwine J H. Amplified RNA synthesized from limited quantities of heterogeneous cDNA. Proc Natl Acad Sci USA. 1990 March; 87(5):1663-7). The said protocol is based on synthesizing a first strand of copy DNA (cDNA) from an oligo-dT 24 bases in length joined to a 20-base fragment of the promoter T7, which recognizes and binds to the poly A strand of the RNA molecule, using reverse transcriptase. Next, the second strand of complementary DNA is generated, followed by amplification starting with the T7 promoter associated with the oligo-dT. This protocol gives good results for transcribing regions of mRNA near to 3′, having an average size of synthesized strand of 1500 nucleotides counting from the 3′ terminal. However, this is not adequate for larger mRNA molecules, as the regions beyond 1500 nucleotides are not amplified and therefore cannot be analyzed.

Another labeling method used in RNA analysis is the FairPlay® III Microarray Labeling Kit (Stratagene, La Jolla, Calif., USA). This system uses a two-step chemical coupling process to fluorescently label the cDNA. Firstly, the nucleotide analog aminoallyl-dNTP is incorporated in the first cDNA strand using reverse transcriptase and random primers, to obtain an amino-modified cDNA. Next, an amino-reactive Cy dye is chemically coupled to the amino-modified cDNA. In this way a labeled cDNA is obtained but without carrying out the amplification of the sample.

Another procedure that is also known is that developed by Rosetta Inpharmatics (Kirkland, Wash., USA) (Castle J, Garrett-Engele P, Armour C D, Duenwald S J, Loerch P M, Meyer M R, Schadt E E, Stoughton R, Parrish M L, Shoemaker D D, Johnson J M. Optimization of oligonucleotide arrays and RNA amplification protocols for analysis of transcript structure and alternative splicing. Genome Biol. 2003; 4(10):R66. Epub 2003 Sep. 19). In this method, starting with an RNA sample, a first stage of synthesizing a first strand of cDNA is performed by reverse transcription using random primers. Next, a second stage of synthesis is carried out of a second cDNA strand using random primers containing a T7 promoter and the double-stranded cDNA obtained is amplified by PCR. Then, an in vitro transcription with T7 RNA polymerase is performed and finally the sample is labeled by reverse transcription using random primers and labeled nucleotide analogs. This method has the disadvantage that it does not satisfactorily cover all regions of any given transcript and that, in addition, the sample is amplified by PCR, which is known to differentially amplify certain particular fragments, rather than other fragments.

### BRIEF SUMMARY OF THE INVENTION

The present invention relates to a method of nucleic acid analysis using composite primers to synthesize a first cDNA strand, synthesis of a second complementary strand, a labeling stage by means of in vitro transcription of the samples using RNA polymerase, and a stage to determine the presence of splicing events in the sample. The method according to the present invention can be used, among other things, for selectively identifying alternative splicing events in the analyzed samples and for the diagnosis of diseases.

In one embodiment, the invention provides a method for providing labeled nucleic acids. The method of this embodiment involves providing or obtaining a sample having RNA. Next, DNA is synthesized from the RNA using oligonucleotides that comprise a random primer portion and a portion having a functional promoter. Double stranded DNA is then synthesized from the single stranded DNA. The double stranded DNA is transcribed into RNA using conditions sufficient for in vitro transcription. The in vitro transcription step utilizes the promoter sequence engineered into the DNA in the earlier steps of this method. The in vitro transcription step serves to label the nucleic acids that are synthesized in this step.

In another embodiment, the invention provides a method for determining the splicing of one or more nucleic acids. The method of this embodiment involves providing or obtaining a sample having RNA. Next, DNA is synthesized from the RNA using oligonucleotides that comprise a random primer portion and a portion having a functional promoter. Double stranded DNA is then synthesized from the single stranded DNA. The double stranded DNA is transcribed into RNA using conditions sufficient for in vitro transcription. The in vitro transcription step can use the promoter sequences engineered into the DNA in the earlier steps of this method. The in vitro transcription step also can serve to label the nucleic acids that are synthesized in this step. The nucleic acids synthesized can then be detected to identify the splicing of the nucleic acid. One method to identify the nucleic acids thus produced is by hybridization to a microarray having probes useful for assessing the alternative splicing of genes.

In yet another embodiment, the invention provides diagnostic and/or prognostic methods.

According to this method, a sample comprising RNA is provided or obtained from a patient that is in need of such an assessment. Next, DNA is synthesized from the sample comprising RNA using oligonucleotides that comprise a random primer portion and a portion having a functional promoter. Double stranded DNA is then synthesized from the single stranded DNA. The double stranded DNA is transcribed into RNA using conditions sufficient for in vitro transcription. The in vitro transcription step can use the promoter sequences engineered into the DNA in the earlier steps of this method. The in vitro transcription step also can serve to label the nucleic acids that are synthesized in this step. The nucleic acid synthesized can then be detected to identify the splicing of the nucleic acid. One method to identify the nucleic acids thus produced is by hybridization to a microarray having probes useful for assessing the alternative splicing of genes. The splicing pattern of the RNA sample can then be compared to a standard (e.g., normal tissue and/or known splicing patterns associated with prognosis or diagnosis) to yield prognostic or diagnostic information. In one embodiment the prognosis and/or method for detecting alternative transcripts may include detecting a splicing pattern associated with cancer. For example, the prognosis and/or method of detecting a splicing pattern can be associated with detecting the splicing pattern and/or alternative transcripts of TMPRSS2 or VEGF.

In still another embodiment, the invention provides a method for determining the splicing of one or more nucleic acids. The method of this embodiment involves providing or obtaining a sample having RNA. Next, DNA is synthesized from the RNA using oligonucleotides that comprise a random primer portion and a portion having a functional promoter and a reverse transcriptase. Double stranded DNA is then synthesized from the single stranded DNA using a primer extension reaction. The double stranded DNA is transcribed into RNA using conditions sufficient for in vitro transcription (e.g., treatment with an RNA polymerase). The in vitro transcription step can use the promoter sequences engineered into the DNA in the earlier steps of this method. The in vitro transcription step also can serve to label the nucleic acids that are synthesized in this step. The nucleic acid synthesized can then be detected to identify the splicing of the nucleic acid. One method to identify the nucleic acids thus produced is by hybridization to a microarray having probes useful for assessing the alternative splicing of genes.

In one embodiment, the invention provides a method for providing labeled nucleic acids. The method of this embodiment involves providing or obtaining a sample having RNA. Next, DNA is synthesized from the RNA using oligonucleotides (a) that comprise (1) a random primer portion and (2) a portion having a functional promoter and oligonucleotides and (b) that comprise (1) a target portion and (2) a portion having a functional promoter. The oligonucleotides (b) have a targeted portion that is used to target a specific gene or genes. The targeted primers can be used to analyze e.g., alternative splicing events where one end of the transcripts is relatively constant and the other end of the transcript is variable e.g., gene fusions, rearrangements, translocations, and deletions. According to this embodiment, double stranded DNA is then synthesized from the single stranded DNA. The double stranded DNA is transcribed into RNA using conditions sufficient for in vitro transcription. The in vitro transcription step utilizes the promoter sequence engineered into the DNA in the earlier steps of this method. The in vitro transcription step serves to label the nucleic acids that are synthesized in this step.

In some embodiments and aspects of the invention, the methods do not involve exponential and/or PCR amplification of the RNA or DNA.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting.

Other features and advantages of the invention will be apparent from the following detailed description, and from the claims.

These and other objects and features of the present invention will become more fully apparent from the following description and appended claims, or may be learned by the practice of the invention as set forth hereinafter.

### BRIEF DESCRIPTION OF THE DRAWINGS

To further clarify the above and other advantages and features of the present invention, a more particular description of the invention will be rendered by reference to specific embodiments thereof which are illustrated in the appended drawings. It is appreciated that these drawings depict only illustrated embodiments of the invention and are therefore not to be considered limiting of its scope. The invention will be described and explained with additional specificity and detail through the use of the accompanying drawings in which:

FIG. 1 shows a detailed diagram of an example of the stages that may be used to carry out one embodiment of the invention. According to this method total mRNA is obtained from a sample (e.g., tissue) and DNA is synthesized (cDNA) from the mRNA by reverse transcription with primers. The primers used are engineered to have a random portion for random priming and a promoter portion that will be used in subsequent steps for in vitro transcription. Next, double stranded DNA is synthesized from the single stranded DNA. The double stranded DNA is then used as a template for labeling via in vitro transcription to give RNA. The RNA can then be analyzed to determine the identity (e.g., sequence, splicing pattern, gene fusions, alternative splicing, etc.).

FIG. 2 shows the results of a synthetic messenger RNA amplification test using composite primers. The triangle data points with solid line represent the Cy3 average for coverage of YOR328W while the circles with the dotted line represents the Cy5 average for the coverage of YOR328W.

FIG. 3 shows the results of a comparison test of labeling a synthetic Saccharomyces mRNA in comparison with the Eberwine method. Square data points with the lighter shade line represent the N6-T7 results using the method of the invention whereas the diamond shape data points with the darker shade line represent the Eberwine oligo dT labeling method.

FIG. 4 shows the results of a comparison test of labeling a synthetic mRNA from the CDC6 gene in comparison with the Eberwine method. The rectangle data points with the lighter shade line represents the N6-T7 results for the average Cy3 and Cy5 value using an example method of the invention whereas the diamond data points with the darker shade line represent the Eberwine method of labeling using oligo-dT method (Cy3 and Cy5 average). This experiment was performed for a single CDC6 isoform.

FIG. 5 shows the structure of the VEGF-189 and VEGF-165 isoforms, as well as the results of hybridization for VEGF of pool 1 versus pool 2. The rectangular data points with the darker shade line represent the VEGF-185 results (pool1 vs. pool 2). The diamond data points with the lighter shade line represent the VEGF 165 results (pool 1 vs. pool 2)

FIG. 6 shows the structure of the VEGF-121 and VEGF-165 isoforms, as well as the results of hybridization for VEGF of pool 1 versus pool 4. The rectangular data points with the darker shade line represent the VEGF-121 results. The diamond data points with the lighter shade line represent the VEGF-165 results.

### DESCRIPTION OF THE INVENTION

The present invention provides kits and methods for labeling polynucleotides and for prognosis and/or diagnosis of disease states of patients. Furthermore, the methods and kits of the invention can be used in research and biomarker discovery applications. In some specific aspects, the inventive methods and kits relate to analyzing splicing and alternative splicing on genes. Generally, the present invention relates to a method of nucleic acid analysis using composite primers to synthesize a first cDNA strand, synthesis of a second complementary strand, a labeling stage by means of in vitro transcription of the samples using RNA polymerase, and a stage to determine the presence of splicing events in the sample. The method according to the present invention can be used, among other things, for selectively identifying alternative splicing events in the analyzed samples and for the diagnosis of diseases.

In one embodiment, the present invention to provide a method of nucleic acid analysis comprising the following stages:

a) synthesis of a first complementary DNA strand (cDNA) from an RNA sample using composite primers comprising a functional promoter sequence and a nonspecific oligonucleotide,
b) synthesis of a second DNA strand, complementary to the cDNA strand obtained in the previous stage, to obtain double-stranded DNA,
c) labeling by in vitro transcription of the double-stranded DNA fragments with an RNA polymerase capable of initiating transcription from the promoter sequence included in the composite primer using a mixture of nucleotides, and
d) determination of the presence of alternative splicing events in the sample.

The term composite primer refers to a primer comprising a functional promoter sequence joined to a nonspecific oligonucleotide having a size of between 5 and 15 nucleotides. The said nonspecific nucleotide can be any nucleotide that has any sequence obtained from all the possible combinations of all the nitrogenated bases that make up a nucleic acid and which, therefore, can recognize and join up with any nucleic acid sequence. In some embodiments the nonspecific oligonucleotide has a size of between 4 and 16 nucleotides.

The term functional promoter sequence refers to a sequence of nucleotides that can be recognized by an RNA polymerase and from which transcription can be initiated. In general, each RNA polymerase recognizes a specific sequence, so that the functional promoter sequence included in the adapters is chosen according to the RNA polymerase used. Examples of RNA polymerases that can be used in the method of the present invention include, but are not limited to, T7 RNA polymerase, T3 RNA polymerase, and SP6 RNA polymerase.

In an embodiment of the invention, the size of the nonspecific oligonucleotide in the composite primer is between 5 and 15 nucleotides.

In an embodiment of the invention, the size of the nonspecific oligonucleotide in the composite primer is of 5, 6, 7, 8, 9, 10, 11, 12, 13, 14, or 15 nucleotides.

In an embodiment of the invention, the size of the nonspecific oligonucleotide in the composite primer is of 6 nucleotides (N6).

In an embodiment of the invention, stage a) is carried out using a temperature gradient of from 25° C. to 42° C.

In an embodiment of the invention, labeling includes incorporation of nucleotide analogs containing directly detectable labeling substances, such as fluorophores, nucleotide analogs incorporating labeling substances detectable in a subsequent reaction, such as biotin or haptenes, or any other type of nucleic acid labeling.

In an embodiment of the invention, the nucleotide analog is selected from among the group comprising Cy3-UTP, Cy5-UTP, fluorescein-UTP, biotin-UTP, and aminoallyl-UTP.

Determination of the presence of alternative splicing events in the sample can be carried out by means of any nucleic acid analysis technique. Microarrays or probes to individual exons and/or splice junctions can be used to determine the splicing of genes of interest.

In one embodiment, the invention provides a method for providing labeled nucleic acids. The method of this embodiment involves providing or obtaining a sample having RNA. Next, DNA is synthesized from the RNA using oligonucleotides (a) that comprise (1) a random primer portion and (2) a portion having a functional promoter and oligonucleotides and (b) that comprise (1) a target portion and (2) a portion having a functional promoter. The oligonucleotides (b) have a targeted portion that is used to target a specific gene or genes. The targeted primers can be used to analyze e.g., alternative splicing events where one end of the transcripts is relatively constant and the other end of the transcript is variable e.g., gene fusions, rearrangements, translocations, and deletions. According to this embodiment, double stranded DNA is then synthesized from the single stranded DNA. The double stranded DNA is transcribed into RNA using conditions sufficient for in vitro transcription. The in vitro transcription step utilizes the promoter sequence engineered into the DNA in the earlier steps of this method. The in vitro transcription step serves to label the nucleic acids that are synthesized in this step.

In one example of this embodiment, the method comprises (1) providing or obtaining a sample comprising RNA (2) synthesizing DNA from the RNA using 2 sets of primers wherein set (a) is comprised of a random portion and a portion having a functional promoter and set (b) is comprised of a primer having a portion that can hybridize to a TMPRSS2 exon and a portion having a function promoter (3) According to this embodiment, double stranded DNA is then synthesized from the single stranded DNA. The double stranded DNA is transcribed into RNA using conditions sufficient for in vitro transcription. The in vitro transcription step utilizes the promoter sequence engineered into the DNA in the earlier steps of this method. The in vitro transcription step serves to label the nucleic acids that are synthesized in this step. The labeled nucleic acid can be detected using any means available to the skilled artisan e.g., microarray, hybridization to specific probes, sequencing etc.

In an embodiment of the invention, determination of the presence of alternative splicing events in the sample is carried out by hybridization of the RNA fragments obtained in stage c) with the immobilized oligonucleotides on a DNA microarray, detection of the labeling incorporated in the fragments to be analyzed, and quantitative comparison of the values of the signals of the hybridized fragments with the values of the reference signals.

In an embodiment of the invention, the immobilized oligonucleotides on the microarray are designed in such a way as to include the sequences corresponding to the splices (e.g., the exon junctions or possible combinations of junctions).

In an embodiment of the invention, the immobilized oligonucleotides on the microarray are designed in such a way that they are located between the sequences corresponding to the splices, i.e. on the sequences corresponding to the exons.

The term microarray or DNA microarray refers to a collection of multiple immobilized oligonucleotides on a solid substrate, where each oligonucleotide is immobilized in a known position so that hybridization with each of the multiple oligonucleotides can be detected separately. The substrate can be solid or porous, planar or non-planar, unitary or distributed. DNA microarrays on which hybridization and detection can be performed can be manufactured with oligonucleotides deposited by any mechanism or with oligonucleotides synthesized in situ by photolithography or by any other mechanism.

It is also an object of the present invention to provide a kit comprising the reagents, enzymes, and additives required to carry out the method of nucleic acid analysis of the invention.

In one embodiment, the invention provides a kit useful for the method of the invention. The kit according to this embodiment comprises (a) instructions for using the kit (b) a component for transcribing RNA into DNA (c) a component for synthesizing double stranded DNA from single stranded DNA and (d) a component for in vitro transcription.

In another embodiment, the invention provides a kit useful for the method of the invention.

In yet another embodiment, the invention provides a kit useful for the method of the invention.

An in vitro transcription component refers to reagents for transcribing DNA into RNA. In one aspect, the component comprises an RNA polymerase. In one aspect, the in vitro transcription component comprises a polymerase capable of transcribing DNA into RNA and rNTPs (e.g., the 5 ribonucleotides needed for transcription. In one specific aspect in vitro transcription component comprises T7 RNA Polymerase, rNTPs, and labeled CTPs. Other RNA polymerases commonly used for in vitro transcription include T3 and S6.

A component capable of synthesizing dsDNA from sDNA refers to an agent that will synthesize double stranded DNA from a single stranded template. In one embodiment, the component comprises a DNA polymerase. In another embodiment, the component comprises primers specific for sequence in the composite primer. In one aspect, the primers will hybridize to a T7 promoter, or complement thereof.

Another object of the present invention is the use of the previously described method for analyzing alternative splicing events in the analyzed sample.

It is also an object of the present invention to use of the previously described method for diagnosing a disease state.

In an embodiment of the invention, the disease state is cancer. In another embodiment of the invention, the disease state is a neurodegenerative disease.

In one embodiment, the method of the invention is used to determine the splicing of at least 1, 2, 3, 4, 5, 6, 7, 8, 9, 10, 11, 12, 13, 14, 15, 20, 25, 30, 35, 40, 45, or 50 tumor suppressors. In one aspect of this embodiment, the one or more tumor suppressors are chosen from p53; the retinoblastoma gene, commonly referred to as Rb1; the adenomatous polyposis of the colon gene (APC); familial breast/ovarian cancer gene I (BRCA1); familial breast/ovarian cancer gene 2 (BRCA2); CDH1 cadherin 1 (epithelial cadherin or E-cadherin) gene; cyclin-dependent kinase inhibitor 1C gene (CDKN1C, also known as p57, KIP2 or BWS); cyclin-dependent kinase inhibitor 2A gene (CDKN2A also known as p16 MTS1 (multiple tumor suppressor 1), TP16 or INK4); familial cylindromatosis gene (CYLD; formerly known as EAC (epithelioma adenoides cysticum)); E1A-binding protein gene (p300); multiple exostosis type 1 gene (EXT1); multiple exostosis type 2 gene (EXT2); homolog of Drosophila mothers against decapentaplegic 4 gene (MADH4; formerly referred to as DPC4 (deleted in pancreatic carcinoma 4) or SMAD4 (SMA- and MAD-related protein 4)); mitogen-activated protein kinase kinase 4 (MAP2K4; also referred to as JNKK1, MEK4, MKK4, or PRKMK4; formerly known as SEK1 or SERK1); multiple endocrine neoplasia type 1 gene (MEN1); homolog of E. coli MutL gene (MLH1 also known as HNPCC (hereditary non-polyposis colorectal cancer) or HNPCC2; formerly referred to as COCA2 (colorectal cancer 2) and FCC2); homolog of E. coli MutS 2 gene (MSH2 also called HNPCC (hereditary non-polyposis colorectal cancer) or HNPCC1 and formerly known as COCA1 (colorectal cancer 1) and FCC1); neurofibromatosis type 1 gene (NF1); neurofibromatosis type 2 gene (NF2); protein kinase A type 1, alpha, regulatory subunit gene (PRKAR1A, formerly known as PRKAR1 or TSE1 (tissue-specific extinguisher 1)); homolog of Drosophila patched gene (PTCH; also called BCNS); phosphatase and tensin homolog gene (PTEN, also called MMAC1 (mutated in multiple advanced cancers 1), formerly known as BZS (Bannayan-Zonana syndrome) and MHAM1 (multiple hamartoma 1)); succinate dehydrogenase cytochrome B small subunit gene (SDHD; also called SDH4); Swi/Snf5 matrix-associated actin-dependent regulator of chromatin gene (SMARCB1, also referred to as BAF47, HSNFS, SNF5/INI1, SNF5L1, STH1P, and SNR1); serine/threonine kinase 11 gene (STK11 also known as LKB1 and PJS); tuberous sclerosis type 1 gene (TSC1 also known as KIAA023); tuberous sclerosis type 2 gene (TSC2, previously referred to as TSC4); von Hipple-Lindau syndrome gene (VHL); and Wilms tumor 1 gene (WT1, formerly referred to as GUD (genitourinary dysplasia), WAGR (Wilms tumor, aniridia, genitourinary abnormalities, and mental retardation), or WIT-2), DAP-kinase, FHIT, Werner syndrome gene, and Bloom syndrome gene. In another aspect, the one or more tumor suppressors are chosen from, APC, BRCA1, BRCA2, CDH1, CDKN2A, DCC, DPC4 (SMAD4), MADR2/JV18 (SMAD2), MEN1, MLH1, MSH2, MTS1, NF1, NF2, PTCH, p53, PTEN, RB1, TSC1, TSC2, VHL, WRN, TMPRSS2, and WT1. In a related embodiment, the invention provides a microarray containing probes for determining the splicing, according to the methods of the invention, the splicing of at least 1, 2, 3, 4, 5, 6, 7, 8, 9, 10, 11, 12, 13, 14, 15, 20, 25, 30, 35, 40, 45, or 50 tumor suppressors. In some aspects, the probes are designed to identify the junctions created by the spliced exons. In some aspects the probes are designed to be specific for the exons.

In one embodiment, the method of the invention is used to determine the splicing of at least 1, 2, 3, 4, 5, 6, 7, 8, 9, 10, 11, 12, 13, 14, 15, 20, 25, 30, 35, 40, 45, or 50 oncogenes. In one aspect, the one or more oncogenes are chosen from K-RAS, H-RAS, N-RAS, EGFR, MDM2, RhoC, AKT1, AKT2, MEK (also called MAPKK), c-myc, n-myc, beta-catenin, PDGF, C-MET, PIK3CA, CDC6, CDK4, cyclin B 1, cyclin D1, estrogen receptor gene, progesterone receptor gene, ERG, a member of the ETS family, ET1, ET4, ErbB1, ErbB2 (also called HER2), ErbB3, ErbB4, TGF-alpha, TGF-beta, ras-GAP, Shc, Nck, Src, Yes, Fyn, Wnt, BCL2, and Bmil. In a related embodiment, the invention provides a microarray containing probes for determining the splicing, according to the methods of the invention, the splicing of at least 1, 2, 3, 4, 5, 6, 7, 8, 9, 10, 11, 12, 13, 14, 15, 20, 25, 30, 35, 40, 45, or 50 oncogenes. In some aspects, the probes are designed to identify the junctions created by the spliced exons. In some aspects the probes are designed to be specific for the exons.

In some embodiments, the method of the invention can be used for determining the splicing of at least 1, 2, 3, 4, 5, 6, 7, 8, 9, 10, 11, 12, 13, 14, 15, 20, 25, 30, 35, 40, 45, or 50 tumorigenic genes. An example of a tumorigenic gene is VEGF. In some aspects, the probes are designed to identify the junctions created by the spliced exons. In some aspects the probes are designed to be specific for the exons.

Thus, in one embodiment, the invention provides a method for detecting VEGF alternative transcripts comprising:

(a) providing an RNA sample obtained from the cells and/or fluid of a VEGF patient;

(b) synthesizing cDNA from the RNA using a composite primer having a random portion and one or more sequences that can be used in later steps for in vitro transcription;

(c) synthesizing double stranded DNA from the cDNA;

(d) transcribing the double stranded DNA into RNA using the sequences engineered into the composite primers;

(e) detecting the splicing patterns of VEGF; to determine the alternative transcripts for VEGF.

In one aspect of this embodiment, the method further comprises analyzing, by the method of the invention, one to fifty tumor suppressors and/or one to fifty oncogenes.

In another embodiment, the invention provides a microarray containing probes for determining the splicing, according to the methods of the invention, the splicing of (A) at least 1, 2, 3, 4, 5, 6, 7, 8, 9, 10, 11, 12, 13, 14, 15, 20, 25, 30, 35, 40, 45, or 50 tumor suppressors; and (B) at least 1, 2, 3, 4, 5, 6, 7, 8, 9, 10, 11, 12, 13, 14, 15, 20, 25, 30, 35, 40, 45, or 50 oncogenes.

In some aspects, the probes and methods are designed to detect alternative transcripts resulting from gene fusions, deletions, and rearrangements associated with a disease state, for diagnosis and/or prognosis. For example, the probes and methods can be designed to detect gene fusions with TMPRSS2 that are associated with aggressive prostate cancer (Nam et al. British Journal of Cancer (2007) 97, 1690-1695; and Hegleson et al. (Cancer Res 2008; 68(1):73-80)). For example, the probes can be designed to detect fusions between TMPRSS2 and ERG, ETV1, ETV4, or ETV5. Identification of these fusions using the methods of the invention in prostate cancer samples can be used for, e.g., predicting prognosis.

In one embodiment, the invention provides a method for determining the prognosis of a prostate cancer patient comprising the steps of:

(a) providing an RNA sample obtained from the cells or fluid of the prostate cancer patient;

(b) synthesizing cDNA from the RNA using a composite primer having a random portion and one or more sequences that can be used in later steps for in vitro transcription;

(c) synthesizing double stranded DNA from the cDNA;

(d) transcribing the double stranded DNA into RNA using the sequences engineered into the composite primers;

(e) detecting the splicing patterns of TMPRSS2; to determine the prognosis of the prostate cancer patient.

In one aspect of this embodiment, the method further comprises analyzing, by the method of the invention, one to fifty tumor suppressors and/or one to fifty oncogenes.

In some aspects of this embodiment, the method comprises detecting the splicing pattern of TMPRSS2 by contacting the RNA synthesized by the method, with probes to one or more exons of TMPRSS2. In some aspects of this embodiment, the method comprises detecting the splicing pattern of TMPRSS2 by contacting the RNA synthesized by the method with probes to one or more exons of a gene selected from ERG, ETV1, and ETV4. In some aspects of this embodiment, the method involves detecting the splicing pattern of TMPRSS2 by contacting the RNA synthesized by the method with probes to one or more splice junctions of exons of TMPRSS2 and one or more exons genes selected from ERG, ETV1, and ETV4.

The method of the present invention is based on synthesizing the first strand of cDNA from an RNA sample using composite primers. In this way, all the RNA molecules present in the original sample can be amplified, regardless of their size. Moreover, the said amplification will be done in proportion to the concentration of each molecule in the original sample. In addition, as the composite primers incorporate the splicing sequence of an RNA-polymerase, it will be possible to transcribe this fragment in vitro for linear amplification and labeling thereof.

Furthermore, in stage a) of the method according to the present invention a temperature gradient of from 25° C. to 42° C. is in addition used to facilitate better matching of the composite primers with the RNA molecule to be amplified.

The aim of the technique is to have the full length of the mRNA homogeneously represented, so that all the exons forming part of an mRNA can be identified with the same signal intensity in the chip.

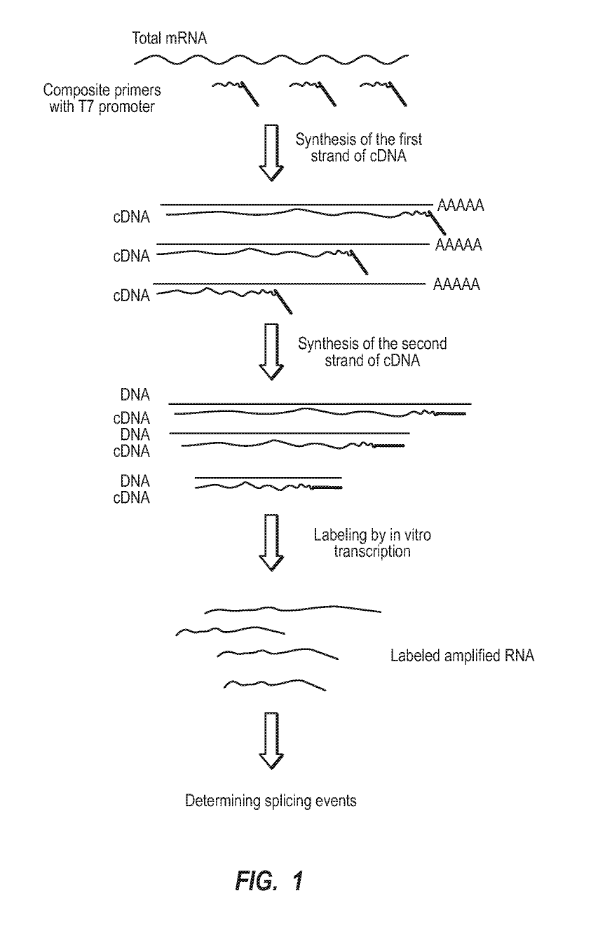

FIG. 1 shows a diagram of the stages constituting an example of the method of the invention.

The method of the present invention enables a plurality of labeled RNAs to be obtained, which in their turn constitute the sample that subsequently can be hybridized using a DNA microarray, which presents certain advantages compared to other methods. In the first place, the RNA-DNA interaction is stronger than the DNA-DNA interaction, enabling an increased average signal intensity to be obtained. In the second place, the single-stranded RNA does not face any competition from complementary molecules present in solution for hybridization on the probes in the microarray surface, so that a greater degree of hybridization is obtained with the probes on the surface of the DNA microarray.

As used herein, the term “probe” refers to any nucleic acid or oligonucleotide that forms a hybrid structure with a sequence of interest in a target gene region (or sequence) due to complementarity of at least one sequence in the probe with a sequence in the target region.

As used herein, the terms “nucleic acid,” “polynucleotide” and “oligonucleotide” refer to nucleic acid regions, nucleic acid segments, primers, probes, amplicons and oligomer fragments. The terms are not limited by length and are generic to linear polymers of polydeoxyribonucleotides (containing 2-deoxy-D-ribose), polyribonucleotides (containing D-ribose), and any other N-glycoside of a purine or pyrimidine base, or modified purine or pyrimidine bases. These terms include double- and single-stranded DNA, as well as double- and single-stranded RNA. A nucleic acid, polynucleotide or oligonucleotide can comprise, for example, phosphodiester linkages or modified linkages including, but not limited to phosphotriester, phosphoramidate, siloxane, carbonate, carboxymethylester, acetamidate, carbamate, thioether, bridged phosphoramidate, bridged methylene phosphonate, phosphorothioate, methylphosphonate, phosphorodithioate, bridged phosphorothioate or sulfone linkages, and combinations of such linkages.

The practice of the present invention employs, unless otherwise indicated, conventional techniques of chemistry, molecular biology, microbiology, recombinant DNA, genetics, immunology, cell biology, cell culture and transgenic biology, which are within the skill of the art. See, e.g., Maniatis, T., et al. (1982) Molecular Cloning: A Laboratory Manual (Cold Spring Harbor Laboratory, Cold Spring Harbor, N.Y.); Sambrook, J., et al. (1989) Molecular Cloning: A Laboratory Manual, 2nd Ed. (Cold Spring Harbor Laboratory, Cold Spring Harbor, N.Y.); Ausubel, F. M., et al. (1992) Current Protocols in Molecular Biology, (J. Wiley and Sons, NY); Glover, D. (1985) DNA Cloning, I and II (Oxford Press); Anand, R. (1992) Techniques for the Analysis of Complex Genomes, (Academic Press); Guthrie, G. and Fink, G. R. (1991) Guide to Yeast Genetics and Molecular Biology (Academic Press); Harlow and Lane (1988) Antibodies: A Laboratory Manual (Cold Spring Harbor Laboratory, Cold Spring Harbor, N.Y.; Jakoby, W. B. and Pastan, I. H. (eds.) (1979) Cell Culture. Methods in Enzymology, Vol. 58 (Academic Press, Inc., Harcourt Brace Jovanovich (NY); Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987).

### EXAMPLES

Below are described some non-exhaustive examples of the method of the present invention.

### Example 1

### Amplification of Synthetic Messenger RNA with Labeling Using Composite Primers

Preparing the DNA

The starting-point was a synthetic DNA 4673 by in length (YOR328W), obtained by a PCR using a direct primer containing a T7 promoter and a reverse primer containing a sequence of 20 thymines (with this reverse primer a fragment comprising 20 adenines was obtained simulating an mRNA).

In Vitro Transcription

100 ng of PCR product were used to carry out the in vitro transcription to RNA from a promoter sequence contained in the direct primer by the addition of 40 U of T7 RNA polymerase (Ambion, USA) and 7.5 mM of rNTPS, the samples being incubated overnight at 37° C. After transcription, the transcribed product was purified using MEGAclear™ columns (Ambion, USA).

Synthesizing the First Strand of Complementary DNA

To 25 ng of synthetic messenger RNA there was added 1.25 μl of N6-T7 composite primers (Thermo Electron, Germany) and the tube was incubated for 10 min at 70° C. followed by 10 min on ice (4° C.). For the amplification, a commercial Message Amp® Kit II from Ambion was used, following the supplier's instructions. To the sample there was added 1 μl of 10× First Strand Buffer+2 μl of dNTP Mix+0.5 μl of RNASe inhibitor, the sample then being incubated at 25° C. for 10 minutes, after which 0.5 μl of ArrayScript reverse transcriptase enzyme was finally added; the entire contents of the tube were homogenized thoroughly and the oven temperature was raised from 25° C. to 42° C. with the samples inside the oven, and incubation continued for 2 hours at 42° C.

Purifying the First Strand of Complementary DNA

After the 2 hours of incubation, the samples were purified through Montage PCR (Millipore) columns to remove the remaining N6 composite primers which may be present in excess quantities in the sample, eluting the sample to a final volume of 20 μl.

Synthesizing the Second Strand of Complementary DNA

To the purified 20 μl there was added 5 μl of 10× Second Strand Buffer (Ambion)+2 μl of dNTP Mix (Ambion)+1 μl of DNA polymerase (Ambion)+0.5 μl of RNAse H (Ambion)+21.5 μl of sterile water to give a total final volume of 30 μl. These reactions were maintained at 16° C. in a water-bath located in a cold room to keep the temperature constant. After incubation these samples were purified by means of DNAclear™ columns (Ambion, USA).

In Vitro Transcription

All of the double-stranded DNA material was used to carry out the in vitro transcription to RNA from a promoter sequence contained in the composite primer (N-6-T7) by the addition of 40 U of T7 RNA polymerase (Ambion, USA) and 7.5 mM of rNTPS, the samples being incubated overnight at 37° C. This reaction was carried out in duplicate, in parallel using Cy3-dUTP or else Cy5-dUTP (Perkin-Elmer, USA) as labeled nucleotides. After transcription the labeled products were purified using MEGAclear™ columns (Ambion, USA).

Microarray Hybridization

500 ng of sample RNA labeled with Cy3 were combined with 500 ng of sample RNA labeled with Cy5 to be hybridized to the oligonucleotide microarray. 100 μl of 2× hybridization solution (Agilent, USA) was added to this RNA mixture and loaded onto the chip exactly as recommended by the company Agilent Technologies. Hybridization took place overnight in a hybridization oven at 60° C. The microarray was subsequently washed with 6× solutions of SSPE+0.005% N-laurylsarcosine (SIGMA) at room temperature for 1 min while stirring, and 0.06× solutions of SSPE+0.005% N-laurylsarcosine at room temperature while stirring to remove any excess of non-hybridized transcripts. Next, the chip was washed for 30 sec in a protective fluorophore solution containing acetonitrile and withdrawn from this solution slowly and at a constant speed to allow the chip to dry thoroughly and uniformly. The intensity signals of each nucleotide in the microarray were detected with an Agilent 62505B scanner.

The amplification of the following fragments was analyzed (INI-XXX indicates that the oligo is located on the said base in the total of 4673 base pairs in the synthetic fragment):

### YOR328W-INI-4318-LEN-39

### YOR328W-INI-4026-LEN-39

### YOR328W-INI-3689-LEN-40

### YOR328W-INI-3351-LEN-40

### YOR328W-INI-2973-LEN-41

### YOR328W-INI-2637-LEN-42

### YOR328W-INI-2217-LEN-42

### YOR328W-INI-1799-LEN-40

### YOR328W-INI-1378-LEN-41

### YOR328W-INI-999-LEN-36

### YOR328W-INI-707-LEN-40

### YOR328W-INI-499-LEN-29

### YOR328W-INI-1,8-LEN-31

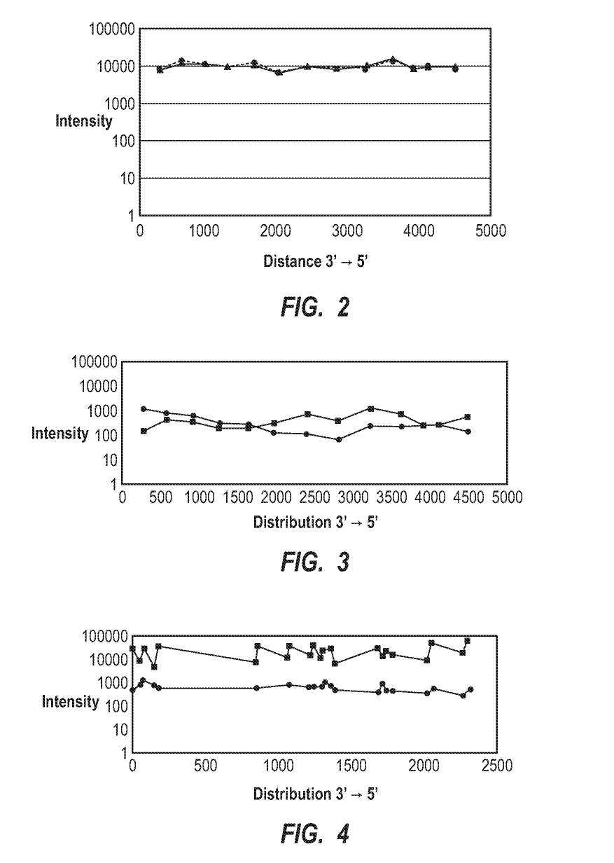

As FIG. 2 shows, all the fragments were represented in the sample within the same range of magnitude.

### Example 2

### Comparison Test of Labeling a Synthetic mRNA of Saccharomyces (Approx. Size 4500 bp) in Comparison with the Eberwine Method

Two labeling tests were conducted in parallel to confirm the greater effectiveness of the method of the invention in comparison to the Eberwine method described earlier. The tests were carried out, with relevant modifications, according to the experimental conditions described in Example 1. In this case, a synthetic mRNA of Saccharomyces about 4500 by in size was used. As regards the primers, an oligo-dT24 primer was used in accordance with the Eberwine method and an N6-T7 composite primer according to the method of the invention.

The result of the detection response for oligonucleotides specific for coverage of 3′ to 5′ when hybridizing the material labeled by the Eberwine method based on an oligo-dT24 primer was compared with that obtained with the method of the invention based on N6-T7 composite primers.

As can be seen from FIG. 3, the results showed that the labeling by means of N6-T7 composite primers allowed homogeneous labeling 3′-->5′ independently of the transcript length and of the distance to 3′ of the oligo; using conventional Eberwine labeling, on the other hand, the intensity decreased as the distance to 3′ increased.

### Example 3

### Comparison Test of Labeling a Synthetic mRNA from the CDC6 Gene (Approx. Size 2300 bp) in Comparison with the Eberwine Method

Two labeling tests were conducted in parallel to confirm the greater effectiveness with respect to the Eberwine method. The tests were carried out, with relevant modifications, according to the experimental conditions described in Example 1. In this case, a synthetic mRNA of CDC6, about 2300 by in size, was used. As regards the primers, an oligo-dT24 primer was used in accordance with the Eberwine method and an N6-T7 composite primer according to the method of the invention Likewise in this case, the starting point was 50 ng of messenger RNA.

The result of the detection response for oligonucleotides specific for coverage of 3′ to 5′ when hybridizing material labeled by the Eberwine method based on an oligo-dT24 primer was compared with that obtained with the method of the invention based on N6-T7 composite primers.

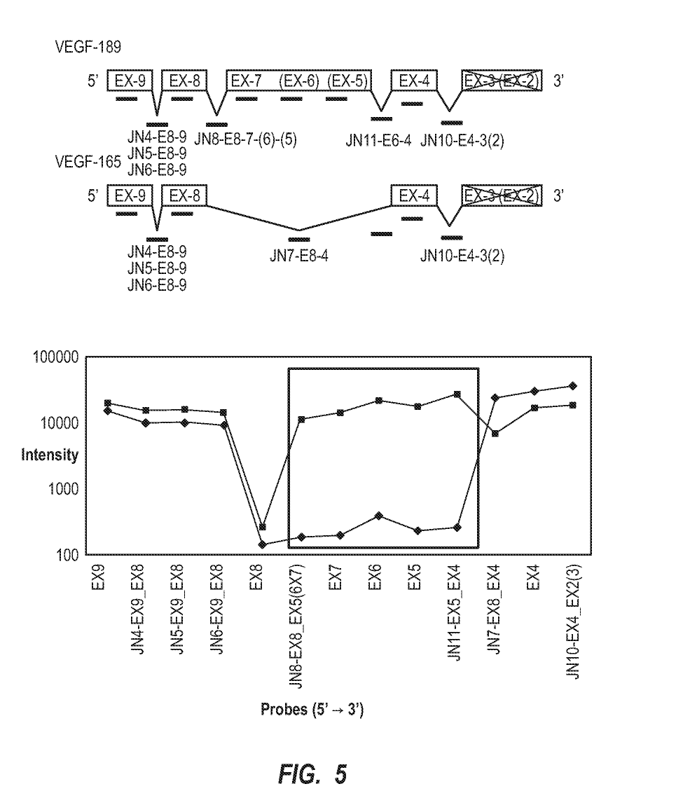

As can be seen from FIG. 4, the results again confirmed that labeling with N6-T7 composite primers allowed homogeneous labeling 3′-->5′ independently of the transcript length and the distance to 3′; using conventional Eberwine labeling, on the other hand, the intensity decreased as the distance to 3′ increased.

### Example 4

### Determining the Splicing Isoforms of the VEGF Gene

An analysis was carried out to determine the capacity of various splicing isoforms for differentiating a gene. The tests were carried out, with relevant modifications, according to the experimental conditions described in Example 1.

For this analysis 3 synthetic transcripts of the VEGF gene were used: VEGF-121 (pool 4), VEGF-165 (pool 1), and VEGF-189 (pool 2).

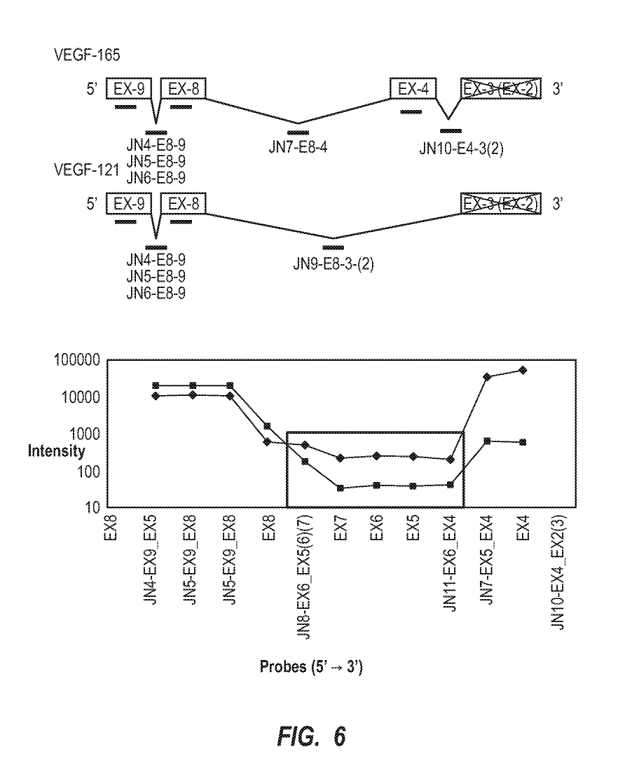

The samples in pool 1 were labeled with Cy3, while the samples in pools 2 and 4 were labeled with Cy5. Moreover, in all the pools the various VEGF isoforms were found to be in equimolar amounts. Hybridizations were carried out to confirm the complete change in form: pool 1 versus pool 2 (FIG. 5) and pool 1 versus pool 4 (FIG. 6).

As can be seen from FIG. 5, the method of the present invention allowed the VEGF-189 form to be differentiated from the VEGF-165 form, which was lacking exons 5 to 7.

Similarly, as FIG. 6 shows, it was also possible to differentiate between isoforms VEGF-165 and VEGF-121, which was lacking exon 4.

In both figures, the boxes are used to indicate the regions in which the various isoforms show differences detectable using the method of the invention.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A method of nucleic acid analysis comprising the following stages:
a) synthesis of a first complementary DNA strand (cDNA) from an RNA sample using composite primers that include functional promoter sequence and a nonspecific oligonucleotide,
b) synthesis of a second DNA strand, complementary to the cDNA strand obtained in the previous stage, to obtain double-stranded DNA,
c) labeling by in vitro transcription of the double-stranded DNA fragments with an RNA polymerase capable of initiating transcription from the promoter sequence included in the composite primer using a mixture of nucleotides, and
d) determination of the presence of alternative splicing events in the sample.

2. The method of nucleic acid analysis as claimed in claim 1, wherein the size of the nonspecific oligonucleotide of the composite primer is between 5 and 15 nucleotides.

3. The method of nucleic acid analysis as claimed in claim 1, wherein the size of the nonspecific oligonucleotide of the composite primer is of 5, 6, 7, 8, 9, 10, 11, 12, 13, 14, or 15 nucleotides.

4. The method of nucleic acid analysis as claimed in claim 1, wherein the size of the nonspecific oligonucleotide of the composite primer is of 6 nucleotides.

5. The method of nucleic acid analysis as claimed in claim 1, wherein stage a) is carried out using a temperature gradient of from 25° C. to 42° C.

6. The method of nucleic acid analysis as claimed in claim 1, wherein the labeling comprises the incorporation of nucleotide analogs containing directly detectable labeling substance.

7. The method of nucleic acid analysis as claimed in claim 6, wherein the directly detectable labeling substance includes one or more of a fluorophore, biotin, haptenes, and/or nucleotide analog selected from the group consisting of Cy3-UTP, Cy5-UTP, fluorescein-UTP, biotin-UTP, aminoallyl-UTP, and combinations thereof.

8. The method of nucleic acid analysis as claimed in claim 1, wherein the RNA polymerase includes one member selected from the group consisting of T7 RNA polymerase, T3 RNA polymerase, and SP6 RNA polymerase.

9. The method of nucleic acid analysis as claimed in claim 1, wherein the determination of the presence of alternative splicing events in the sample is carried out by hybridization of the RNA fragments obtained in stage c) with the immobilized oligonucleotides on a DNA microarray, detection of the labeling incorporated in the fragments to be analyzed, and quantitative comparison of the signal values of the hybridized fragments with the values of the reference signals.

10. The method of nucleic acid analysis as claimed in claim 9, wherein the immobilized oligonucleotides on the microarray are designed in such a way as to include the sequences corresponding to the splices.

11. The method of nucleic acid analysis as claimed in claim 9, wherein the immobilized oligonucleotides on the microarray are located between the sequences corresponding to the splices.

12. A kit comprising the reagents, enzymes, and additives required to carry out the method of nucleic acid analysis as claimed in claim 1.

13. A method comprising, diagnosing a disease state using the method of claim 1.

14. A method as in claim 13, wherein the disease state is cancer.

15. A method as in claim 13, wherein the disease state is a neurodegenerative disease.

16. A method for determining the prognosis of a prostate cancer patient comprising the steps of:
(a) providing an RNA sample obtained from cells and/or fluid of the prostate cancer patient;
(b) synthesizing cDNA from the RNA sample using a composite primer having a random portion and one or more sequences that can be used in later steps for in vitro transcription;
(c) synthesizing double stranded DNA from the cDNA;
(d) transcribing the double stranded DNA into RNA using the sequences engineered into the composite primers;
(e) detecting the splicing patterns of TMPRSS2 to determine the prognosis of prostate cancer patient.

17. The method of claim 16, wherein said detecting the splicing pattern of TMPRSS2 comprised contacting the RNA with probes to one or more exons of TMPRSS2.

18. The method of claim 16, wherein said detecting the splicing pattern of TMPRSS2 comprises contacting the RNA with probes to one or more exons of a gene selected from ERG, ETV1, and ETV4.

19. The method of claim 16, wherein said detecting the splicing pattern of TMPRSS2 comprises contacting the RNA with probes to one or more splice junctions of exons of TMPRSS2 and one or more exons genes selected from ERG, ETV1, and ETV4.

20. A method for detecting VEGF alternative transcripts comprising the steps of:
(a) providing an RNA sample obtained from cells and/or fluid of the VEGF patient;
(b) synthesizing cDNA from the RNA sample using a composite primer having a random portion and one or more sequences that can be used in later steps for in vitro transcription;
(c) synthesizing double stranded DNA from the cDNA;
(d) transcribing the double stranded DNA into RNA using the sequences engineered into the composite primers;
(e) detecting the splicing patterns of VEGF; to determine the alternative transcripts for VEGF.

21. The method of claim 21 further comprising detecting the splicing pattern of one or more additional markers according to steps (a)-(e).
